(19) United States
(12) Patent Application Publication
(10) Pub. No.: US 2024/0315618 A1
(43) Pub. Date: Sep. 26, 2024

(54) Sample Container for Capillary Blood Collection

(71) Applicant: Becton, Dickinson and Company, Franklin Lakes, NJ (US)

(72) Inventors: Anthony V. Torris, Montclair, NJ (US); Kishore K. Bokka Srinivasa Rao, Ridgewood, NJ (US); Charles Peter Althoff, West Nyack, NY (US); Vlad Yakhnich, Park Ridge, NJ (US)

(21) Appl. No.: 18/574,797

(22) PCT Filed: Jun. 23, 2022
(86) PCT No.: PCT/US22/34643

§ 371 Date: Dec. 28, 2023

Related U.S. Application Data
(60) Provisional application No. 63/216,277, filed on Jun. 29, 2021.

(51) Int. Cl. A61B 5/15

(52) U.S. Cl. CPC A61B 5/150022; A61B 5/150343; A61B 5/150351; A61B 5/150748

(57) ABSTRACT

A sample collection container configured to be removably mounted to a blood collection device includes a container body and a lid. The container body defines an interior configured to receive a sample provided from the blood collection device. The container body includes an upper portion having an open top and a lower portion having a closed bottom. The lid includes: a lid body; a flexible connection connected between the lid body and the upper portion of the container body that moves the lid between a closed position and an open position; a pierceable septum mounted to the lid body that seals the upper portion open top when the lid is in the closed position; and a latch. The latch extends from the lid body and engages a catch on the upper portion of the container body to secure the lid in the closed position.

### CROSS-REFERENCE TO RELATED APPLICATION

The present application claims priority to U.S. Provisional Application Ser. No. 63/216,277, filed Jun. 29, 2021, entitled “Sample Container for Capillary Blood Collection”, the entire disclosure of which is hereby incorporated by reference in its' entirety.

### BACKGROUND OF THE INVENTION

### Field of the Invention

The present disclosure relates generally to collection containers for containing a biological sample, such as a blood sample. More particularly, the present disclosure relates to a sample collection container configured to connect to and receive a blood sample from a finger-based capillary blood collection device including features for ensuring that the container remains appropriately closed and sealed to avoid leakage and/or contamination of the blood sample.

### Description of Related Art

Devices for obtaining and collecting biological samples, such as blood samples, are commonly used in the medical industry. One type of blood collection that is commonly done in the medical field is capillary blood collection, which is often done to collect blood samples for testing. Certain diseases, such as diabetes, require that a patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed, which are used for puncturing the skin of a patient, to obtain a capillary blood sample from the patient.

Many different types of lancet devices are commercially available to hospitals, clinics, doctors' offices, and the like, as well as to individual consumers. Such devices typically include a sharp-pointed member, such as a needle, or a sharp-edged member, such as a blade, that is used to make a quick puncture wound or incision in the patient's skin in order to provide a small outflow of blood. In order to simplify capillary blood collection, lancet devices have evolved into automatic devices that puncture or cut the skin of the patient upon actuation of a triggering mechanism. In some devices, the needle or blade is kept in a standby position until it is triggered by the user. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releasing the lever from interference engagement with the lancet structure. The blood sample that is received is then collected and/or tested. This testing can be done by a Point-of-Care (POC) testing device or it can be collected and sent to a testing facility.

Use of lancet devices for capillary blood collection can be complex requiring a high skill level for the healthcare worker performing the blood collection procedure. The multi-step nature of the capillary blood collection process can introduce several variables that may cause sample quality issues, such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

In view of such common sources of process variability and associated sample quality issues, there is a need for blood collection devices and associated containers for securely receiving and storing biological samples in sealed containers with minimal risk of leakage or contamination. In particular, the collection containers of the present disclosure are configured to receive fluid samples from blood collection devices and to retain the received fluid samples in a securely sealed container until ready for use. Further, the containers of the present disclosure are configured to be easy to use, meaning that the containers are easy to close or otherwise manipulate without requiring users to exert substantial force on the container or blood collection devices during use.

### SUMMARY OF THE INVENTION

According to an aspect of the disclosure, a sample collection container configured to be removably mounted to a blood collection device includes a container body and a lid. The container body defines an interior configured to receive a sample provided from the blood collection device. The container body includes an upper portion having an open top and a lower portion having a closed bottom. The lid includes: a lid body; a flexible connection between the lid body and the upper portion of the container body that moves the lid between a closed position, in which the lid covers the open top of the upper portion, and an open position, where the open top of the upper portion is uncovered; a pierceable septum mounted to the lid body that seals the upper portion of the open top when the lid is in the closed position; and a latch. The latch extends from the lid body and engages a catch on the upper portion of the container body to secure the lid in the closed position.

According to another aspect of the disclosure, a blood collection container includes a container body having a container body closed bottom, a container body open top, and a container body annular sidewall extending therebetween. The container further includes a cap that is separable from the container body. The cap includes a cap open bottom removably inserted over the container body open top, a cap open top, and a cap annular sidewall extending therebetween. The container further includes a lid removably mounted over the cap open top. The cap is sized to be press fit over the container body and, when fully seated on the container body, the cap is restricted from rotation relative to the container body.

According to another aspect of the disclosure, a blood collection assembly includes: a finger holder having a finger receiving portion and an actuation portion; a container engagement portion connected to the holder; and a collection container removably connectable to the container engagement portion. The container includes a container body defining an interior configured to receive a sample provided from the blood collection device, and a lid. The container body includes an upper portion comprising an open top and a lower portion comprising a closed bottom. The lid includes: a lid body; a flexible connection between the lid body and the upper portion of the container body that moves the lid between a closed position, in which the lid covers the open top of the upper portion and an open position, where the open top of the upper portion is uncovered; a pierceable septum mounted to the lid body that seals the upper portion open top when the lid is in the closed position; and a latch extending from the lid body that engages a catch on the upper portion of the container body to secure the lid in the closed position.

According to another aspect of the disclosure, a cap removal assembly for disassembly of a cap of a blood collection container from a container body of the blood collection container includes: a stand configured to support the blood collection container in an upright position and to restrict rotation of the container relative to the stand and a tool. The tool includes a cap remover and a handle extending therefrom. The cap remover includes a body defining an opening or recess sized to receive a cap of the blood collection container, and at least one protrusion extending from the body into the opening or recess positioned to engage a portion of the cap, such that rotation of the tool relative to the cap releases the cap from a body of the container.

Non-limiting illustrative examples of embodiments of the present disclosure will now be described in the following numbered clauses:

Clause 1: A sample collection container configured to be removably mounted to a blood collection device, the collection container comprising: a container body defining an interior configured to receive a sample provided from the blood collection device, the container body comprising an upper portion comprising an open top and a lower portion comprising a closed bottom; and a lid comprising: a lid body; a flexible connection between the lid body and the upper portion of the container body that moves the lid between a closed position, in which the lid covers the open top of the upper portion, and an open position, where the open top of the upper portion is uncovered; a pierceable septum mounted to the lid body that seals the upper portion open top when the lid is in the closed position; and a latch extending from the lid body that engages a catch on the upper portion of the container body to secure the lid in the closed position.

Clause 2: The collection container of clause 1, wherein the latch comprises a frame formed from a first material and a cushion formed from a second material that is less rigid than the first material, wherein the cushion is positioned proximate to a leading edge of the frame of the lid and is configured to restrict the latch from being disengaged from the catch.

Clause 3: The collection container of clause 2, wherein the frame comprises an opening that receives the catch of the upper portion when the lid is in the closed position, and wherein the cushion is positioned on the frame between the opening and the leading edge of the frame.

Clause 4: The collection container of clause 2 or clause 3, wherein the lid body, the flexible connection, and the container body comprise the first material, and wherein the septum comprises the second material.

Clause 5: The collection container of any of clauses 2-4, wherein the first material is a semi-rigid plastic, such as polypropylene, and the second material is a thermoplastic elastomer, such as liquid silicone rubber (LSR), urethane rubber, and latex rubber.

Clause 6: The collection container of any of clauses 1-5, wherein the catch on the upper portion of the container body comprises a sloped surface positioned such that, as the lid transitions between the open position and the closed position, the latch deflects over the catch prior to engaging the catch.

Clause 7: The collection container of any of clauses 1-6, wherein the septum comprises an inwardly facing surface that faces the interior of the container body and an outwardly facing surface opposite the inwardly facing surface, the outwardly facing surface comprising a recess or well positioned to collect any fluid that escapes from the interior of the container body during retraction of a needle through the septum.

Clause 8: The collection container of any of clauses 1-7, wherein the lid body comprises an annular flange defining an opening and wherein the septum is inserted through the opening with the annular flange received within an annular notch of the septum.

Clause 9: The collection container of any of clauses 1-8, wherein the septum comprises an inwardly facing surface that faces the interior of the container body when the lid is in the closed position, the inwardly facing surface of the septum comprising a chamfered peripheral edge.

Clause 10: The collection container of clause 9, wherein the inwardly facing surface of the septum defines a central cavity, which enhances radial deformation of the septum during centrifugation.

Clause 11: The collection container of clause 10, wherein the central cavity comprises a tapered annular surface.

Clause 12: The collection container of any of clauses 1-11, wherein the upper portion open top comprises an annular chamfered edge positioned to contact a chamfered peripheral edge of the septum as the lid moves to the closed position.

Clause 13: The collection container of clause 12, wherein the annular chamfered edge of the upper portion deforms the septum when the lid is in the closed position, thereby scaling the interior of the container body.

Clause 14: The collection container of clause 13, wherein the septum is configured to radially expand during centrifugation, thereby enhancing the sealing of the interior of the container body.

Clause 15: The collection container of any of clauses 1-14, wherein the lower portion of the container body and the upper portion of the container body are separable and removably attached together.

Clause 16: The collection container of clause 15, wherein the upper portion is sized to be press fit over the lower portion and, when fully seated on the lower portion, the upper portion is restricted from rotation relative to the lower portion.

Clause 17: The collection container of any of clauses 1-16, wherein the lower portion of the container body comprises the lower portion closed bottom, a lower portion open top, and a lower portion annular sidewall extending therebetween, and wherein the upper portion of the container body comprises an upper portion open bottom inserted over the lower portion open top of the container body, the upper portion open top, and an upper portion annular sidewall extending therebetween.

Clause 18: The collection container of clause 17, wherein the lower portion of the container body comprises an annular flange extending radially outwardly from an outer surface of the lower portion annular sidewall, and wherein, when the upper portion is inserted onto the lower portion, the upper portion open bottom contacts the annular flange of the lower portion to restrict rotation of the upper portion relative to the lower portion.

Clause 19: The collection container of clause 18, wherein the annular flange extending from the lower portion and the upper portion open bottom comprise corresponding ramp surfaces configured to be axially aligned when the upper portion is inserted onto the lower portion.

Clause 20: The collection container of clause 19, wherein rotation of the upper portion relative to the lower portion causes the ramp surface of the upper portion to slide along the ramp surface of the annular flange of the lower portion, thereby lifting the upper portion off of the lower portion.

Clause 21: The collection container of any of clauses 17-20, wherein the lower portion comprises an annular protrusion extending radially outward from the outer surface of the lower portion sidewall, and wherein an inner surface of the upper portion sidewall comprises an annular recess that receives the annular protrusion of the lower portion.

Clause 22: The collection container of clause 21, wherein the annular protrusion extending from the lower portion comprises a sloped upper surface, a substantially planar lower surface, and a cylindrical peripheral edge extending therebetween.

Clause 23: The collection container of any of clauses 17-22, wherein an inner surface of the lower portion sidewall comprises a tapered portion that tapers radially inwardly from the lower portion open top.

Clause 24: The collection container of clause 23, wherein there is tapered interference fit between the outer surface of the lower portion sidewall and the inner surface of the upper portion sidewall.

Clause 25: The collection container of any of clauses 1-24, wherein the flexible connection comprises a hinge.

Clause 26: A blood collection container comprising: a container body comprising a container body closed bottom, a container body open top, and a container body annular sidewall extending therebetween; a cap that is separable from the container body, the cap comprising a cap open bottom removably inserted over the container body open top, a cap open top, and a cap annular sidewall extending therebetween; and a lid removably mounted over the cap open top, wherein the cap is sized to be press fit over the container body and, when fully seated on the container body, the cap is restricted from rotation relative to the container body.

Clause 27: A blood collection assembly comprising: a finger holder comprising a finger receiving portion and an actuation portion; a container engagement portion connected to the holder; and a collection container removably connectable to the container engagement portion, the container comprising a container body defining an interior configured to receive a sample provided from the blood collection device, the container body comprising an upper portion comprising an open top and a lower portion comprising a closed bottom, and a lid comprising: a lid body; a flexible connection between the lid body and the upper portion of the container body that moves the lid between a closed position, in which the lid covers the open top of the upper portion and an open position, where the open top of the upper portion is uncovered; a pierceable septum mounted to the lid body that seals the upper portion open top when the lid is in the closed position; and a latch extending from the lid body that engages a catch on the upper portion of the container body to secure the lid in the closed position.

Clause 28: A cap removal assembly for disassembly of a cap of a blood collection container from a container body of the blood collection container, the cap removal assembly comprising: a stand configured to support the blood collection container in an upright position and to restrict rotation of the container relative to the stand; and a tool comprising a cap remover and a handle extending therefrom, the cap remover comprising a body defining an opening or recess sized to receive a cap of the blood collection container, and at least one protrusion extending from the body into the opening or recess positioned to engage a portion of the cap, such that rotation of the tool relative to the cap releases the cap from a body of the container.

Clause 29: The cap removal assembly of clause 28, wherein once the cap is released from the body, the cap can be removed from the body by lifting the cap axially from the body.

Clause 30: The cap removal assembly of clause 28 or clause 29, wherein the body comprises a ring defining the opening, and wherein the cap can be inserted through the opening when the tool is in either an upwardly facing orientation or a downwardly facing orientation.

Clause 31: The cap removal assembly of clause 30, wherein the tool permits right handed operation when in the upwardly facing orientation and left handed operation when in the downwardly facing orientation.

Clause 32: The cap removal assembly of any of clauses 28-31, wherein the tool further comprises a pressing member mounted to the handle and comprising a distal end extending over the opening of the removal portion, the pressing member being positioned to press against a top of the cap when the cap is inserted through the opening, thereby preventing the container from lifting out of the stand when the tool and cap are rotated relative to the container body.

Clause 33: The cap removal assembly of any of clauses 28-32, wherein the cap remover comprises two protrusions extending into the opening or recess.

Clause 34: The cap removal assembly of clause 33, wherein the two protrusions are positioned such that a virtual line passing between the two protrusions through a center of the opening or recess is sloped relative to a longitudinal axis of the handle.

Clause 35: The cap removal assembly of clause 34, wherein the slope is from about 5 degrees to about 25 degrees, preferably about 15 degrees.

### BRIEF DESCRIPTION OF THE DRAWINGS

FIG. 1A is a perspective view of a capillary blood collection device for obtaining a blood sample from a patient's finger and a collection container, according to an aspect of the present disclosure.

FIG. 1B is a cross-sectional view of a capillary blood collection device and lancet, according to an aspect of the present disclosure.

FIG. 1C is a perspective view of a holder of a capillary blood collection device, according to an aspect of the present disclosure.

FIG. 1D is a schematic drawing showing a top view of the holder of FIG. 1C connected to a patient's finger for performing a blood collection procedure.

FIG. 1E is another schematic drawing showing a front view of the holder of FIG. 1C connected to the patient's finger.

FIG. 2A is a perspective view of a top portion of a sample collection container with the lid in the open position, according to an aspect of the present disclosure.

FIG. 2B is a perspective view of the top portion of the sample collection container of FIG. 2A with the lid in the closed position.

FIG. 2C is a cross-sectional view of the top portion of the sample collection container of FIG. 2A with the lid in the closed position, according to an aspect of the present disclosure.

FIGS. 3A and 3B show results from finite element analysis modeling showing a scaling force for a septum of a blood collection device after assembly and with high fluid pressure, according to aspects of the present disclosure.

FIG. 4A is a perspective view of a sample collection container with the upper portion or cap attached to the lower portion, according to an aspect of the present disclosure.

FIG. 4B is a perspective view of the sample collection container of FIG. 4A with the upper portion or cap separated from the lower portion of the sample collection container.

FIG. 4C is a cross-sectional view of the sample collection container of FIG. 4A with the upper portion or cap attached to the lower portion of the container.

FIG. 4D is an enlarged cross section view of a portion of the sample collection container of FIG. 4C showing the interface between the upper portion or cap and the lower portion of the container body, according to an aspect of the present disclosure.

FIG. 5A is a perspective view of a cap removal assembly and sample collection container, according to aspects of the present disclosure.

FIG. 5B is a cross-sectional view of the cap removal assembly of FIG. 5A.

FIG. 5C is a perspective view of a tool of the cap removal assembly of FIG. 5A, according to an aspect of the present disclosure.

FIG. 5D is a top view of the tool of FIG. 5C showing positioning of the protrusions of the cap remover, according to an aspect of the present disclosure.

FIG. 5E is a schematic drawing showing use of the tool to release an upper portion or cap of the sample collection container from the lower portion of the container.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure relates to blood collection devices 10 or assemblies configured to collect a capillary blood sample and including a sample collection container 16 for receiving the blood sample. The sample collection container 16 can be used for centrifuging the sample. In particular, the present disclosure is directed to closures for the container 16, which can include, for example, a cap and integrated lid. The cap and integrated lid are configured to provide a sufficiently robust closure to maintain an appropriate seal for the container 16 during and after centrifugation. The container 16 can be made from a rigid or semi-rigid material, such as polypropylene. The integrated lid can be made from a mixture of a rigid or semi-rigid material and a flexible material, such as a thermoplastic elastomer. The materials and geometry of the lid are selected to allow for one-handed closure and initial sealing of the container 16, meaning that the lid can be closed easily with little required force. The closure should also provide a high sealing pressure during centrifugation to prevent leaking. In some examples, the centrifugation process can be a reverse centrifugation process, in which the container 16 is inserted into the centrifuge in an upside-down orientation (i.e., with the lid of the container 16 positioned in the bottom of a receptacle receiving cavity or well of the centrifuge). Accordingly, the lid can be designed to withstand forces exerted on the lid during reverse centrifuging. In some examples, a latch is provided to secure the lid in the closed position and to ensure proper insertion-depth of a flexible septum or sealing element of the lid.

The present disclosure is also directed to a two-piece sample collection container 16 formed from a lower portion or container body and an upper portion or cap. The two portions can be separable and connected together at a joining interface. The joining interface between the portions of the container body can be configured to allow for simple assembly of the container body, while also providing a secure air-tight seal between the portions of the container body. It is also important to allow for controlled removal of the upper portion or cap from the lower portion of the container body so that the fluid contents of the collection container 16 are accessible for analysis. For example, as described in further detail herein, the upper portion and/or the lower portion of the container body can include ramp or cam surfaces arranged such that rotation of the upper portion relative to the lower portion causes the upper portion or cap to separate from the lower portion of the container body.

### Blood Collection Assembly or Device

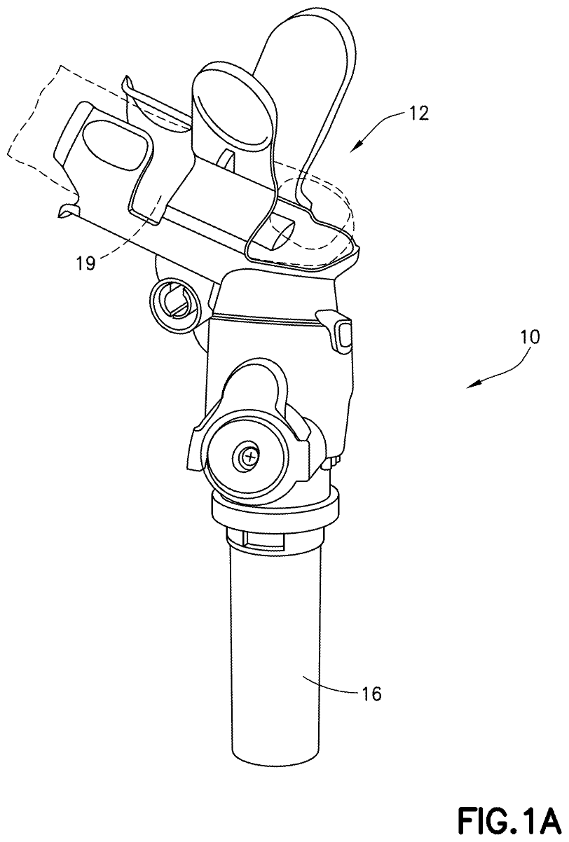

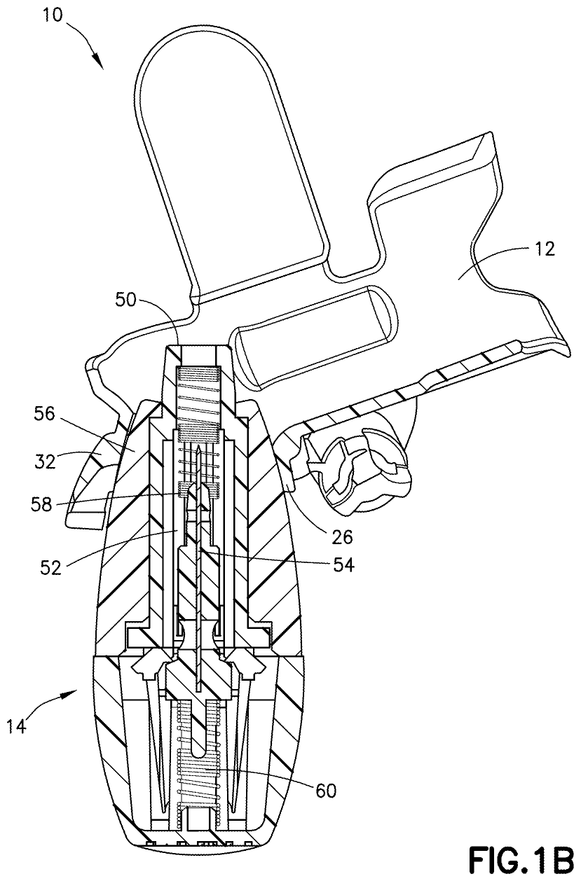

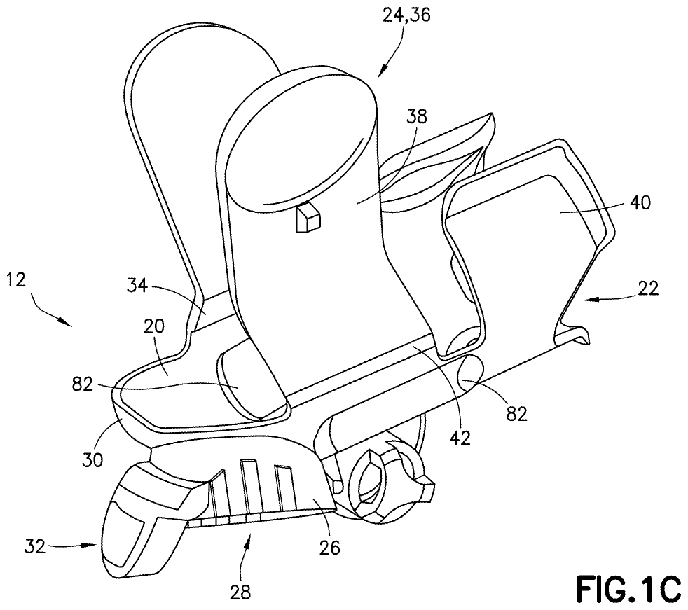

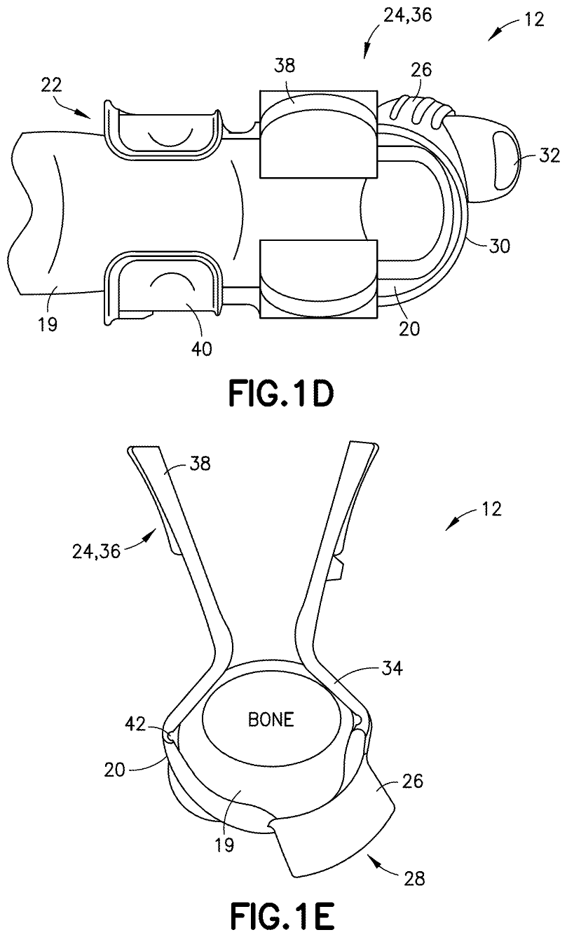

Examples of blood collection devices 10 or assemblies including features of the present disclosure are shown in FIGS. 1A-1E. The blood collection devices 10 can be, for example, a self-contained and fully integrated finger-based capillary blood collection device with the ability to lance, collect, and stabilize a high volume capillary blood sample, e.g., up to or above 500 microliters, in the sample collection container 16. The blood collection device 10 or assembly can also be formed from separable components (i.e., a finger cuff, lance, and reservoir) that can be connected and/or used together to obtain a blood sample. Other exemplary capillary blood collection devices and assemblies that can be modified to include features of the present disclosure are described, for example, in United States Patent Appl. Pub. No. 2019/0216380, entitled “Device for Obtaining a Blood Sample”; United States Patent Appl. Pub. No. 2019/0223772, entitled “Device for the Attached Flow of Blood”; and PCT Publication No. WO 2020/167746, entitled “Capillary collector with rotatable connection,” each of which is incorporated herein by reference in its entirety.

With reference to FIGS. 1A and 1B, the example blood collection device 10 includes an integrated holder 12, a lancet housing or lancet 14 (shown in FIG. 1B) for puncturing a finger 19 (shown in FIGS. 1D and 1E) of the patient, and the sample collection container 16 (shown in FIGS. 1A and 2A-3D). In other examples, the blood collection device 10 can be provided as a semi-integrated device 10 including, for example, an integrated lancet housing 14 and collection container 16 that can be connected with a separate holder 12. In other examples, a semi-integrated device 10 may have an in-line flow and an integrated lancet housing 14 and collection container 16, which can be connected with a separate holder 12.

The holder 12 is configured to receive a sample source, e.g., the finger 19 of a patient, for supplying a biological sample, such as a blood sample. As shown in FIGS. 1C-1E, the holder 12 generally includes a finger receiving portion 20 having a first opening 22, an actuation portion 24, a port 26 having a second opening 28, and a finger end guard 30. In some examples, the finger end guard 30 provides a stop portion for properly aligning and securing the finger 19 within the holder 12. The finger end guard 30 further assists in ensuring the patient's finger 19 is placed at a proper position within the finger receiving portion 20 so that applied pressure to the patient's finger 19 will result in adequate blood flow. The finger end guard 30 can have a curved fingertip rest that ensures the patient's finger 19 stops at an end of the finger receiving portion 20, while permitting the patient's finger nail to clear the end of the finger receiving portion 20. The finger receiving portion 20 permits use of the holder 12 with artificial and natural fingernail styles present in the patient population.

The first opening 22 of the finger receiving portion 20 is configured for receiving the sample source, e.g., the finger 19. The sample source may also include other parts of the body capable of fitting within the first opening 22, such as toes or other extremities. The port 26 is in communication with the finger receiving portion 20. For example, with a finger 19 received within the holder 12, the port 26 is in communication with a portion of the finger 19. The second opening 28 of the port 26 is configured for receiving the lancet housing or lancet 14 (shown in FIG. 1B) and the collection container 16 (shown in FIGS. 1A and 2A-3D). In some examples, the port 26 further includes a locking portion 32 for securely receiving the lancet housing or lancet 14 and the collection container 16 within the port 26.

The actuation portion 24 of the device 10 is transitionable between a first position, in which the holder 12 defines a first diameter, and a second position, in which the holder 12 defines a second diameter, with the second diameter being less than the first diameter. Further, in the first position, the holder 12 defines a first elliptical shape. In the second position, the holder 12 defines a second elliptical shape, with the first elliptical shape being different than the second elliptical shape. In this manner, with the holder 12 in the second position with a reduced diameter, a portion of the holder 12 contacts the sample source (i.e., the finger 19) and the actuation portion 24 of the holder 12 is able to pump and/or extract blood, as described in more detail below.

In some examples, the actuation portion 24 includes a contact member 34. With the actuation portion 24 in the first position, the contact member 34 is in a disengaged position, i.e., the contact member 34 is provided in a first position with respect to the sample source, such that the contact member 34 may be in slight contact therewith. With the actuation portion 24 in the second position, the contact member 34 is in an engaged position, i.e., the contact member 34 is provided in a second position with respect to the finger 19, such that the contact member 34 is in an applied pressure contact with the finger 19, and the actuation portion 24 of the holder 12 is able to pump and/or extract blood. For example, with the contact member 34 in the engaged position, the contact member 34 exerts a pressure on the sample source.

In some examples, the actuation portion 24 includes a pumping member 36 for applying pressure to the finger 19, such as a pair of opposed tabs or wings 38. Each wing 38 can include a contact member 34. The holder 12 can also include a living hinge portion 42. The living hinge portion 42 allows the user to squeeze the wings 38 between a first position (passive state) and a second position (active state). It is believed that use of the tabs or wings 38 to draw blood out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12, while flexing to accommodate the largest patient fingers within a holder 12 without blood occlusion. In some examples, the wings 38 may be positioned on the finger receiving portion 20 at a position located proximal of a patient's fingernail and distal of a patient's first knuckle to avoid hard tissues on the patient's finger 19.

The holder 12 can be configured to allow a user to repeatedly squeeze and release the wings 38 to pump and/or extract blood from a finger 19 until a desired amount of blood is filled in the collection container 16. The wings 38 are configured to flex to maintain gentle contact with a range of patient finger sizes that may be used with the holder 12 and to retain the holder 12 on the patient's finger 19. The wings 38 may also provide active pressure features for the holder 12.

In some examples, the holder 12 can include a stability extension portion 40. This provides additional support for the holder 12 to be securely placed onto the finger 19. In one example, the finger receiving portion 20 forms a generally C-shaped member and includes a plurality of inner gripping members for providing additional grip and support for the holder 12 to be securely placed onto a patient's finger 19. The stability extension portion 40 assists in maintaining contact with the patient's finger 19 during use of the holder 12 while avoiding the blood supply and knuckles of the patient's finger 19.

The blood collection device 10 for obtaining the blood sample also includes the lancet housing or lancet 14 (shown in FIG. 1B) that is removably connectable to the port 26 of the holder 12. Referring to FIG. 1B, the lancet housing or lancet 14 can include an inlet or opening 50, an interior 52, a puncturing element 54, an engagement portion 56, a retractable mechanism 58, and a drive spring 60. The puncturing element 54 can be moveable between a pre-actuated position, wherein the puncturing element 54 is retained within the interior 52 of the lancet housing 14, and a puncturing position, wherein at least a portion of the puncturing element 54 extends through the inlet 50 of the lancet housing or lancet 14 to lance a portion of a finger 19. In one example, the lancet 14 of the present disclosure is a contact activated lancet and may be constructed in accordance with the features disclosed in U.S. Pat. No. 9,380,975, entitled “Contact Activated Lancet Device”, which is incorporated herein by reference in its entirety.

In some examples, the holder 12 and the lancet housing or lancet 14 are separate components that can be removably connectable to the port 26 of the holder 12. In such examples, the lancet housing or lancet 14 includes the engagement portion 56. The lancet housing or lancet 14 can be pushed into the port 26 of the holder 12, such that the engagement portion 56 of the lancet housing or lancet 14 is locked within the locking portion 32 of the holder 12. In this manner, the lancet housing 14 is securely connected and locked to the holder 12, such that the puncturing element 54 of the lancet housing 14 can be activated to lance or puncture a sample source, e.g., the finger 19. In some examples, the port 26 of the holder 12 includes a plurality of ribs for securing and locking the lancet 14 or the collection container 16 in the port 26.

To activate the lancet 14, the lancet 14 is pushed against the finger 19 to activate the retractable mechanism 58 and drive spring 60 of the lancet 14 to lance the finger 19. After puncturing, the puncturing element 54 is immediately retracted and safely secured within the interior 52 of the lancet housing 14. Once the finger 19 is punctured, the blood sample is squeezed from the finger 19 into the collection container 16. The collection container 16 may also contain a sample stabilizer, e.g., an anticoagulant, to stabilize a blood sample and/or a component of a blood sample disposed therein. The collection container 16 may also include at least one fill line(s) corresponding to a predetermined volume of sample. The collection container 16 may also indicate/meter a collected volume of blood.

In order to use the capillary blood collection device 10 shown in FIGS. 1A-1E, a desired finger 19 is first cleaned and a holder 12 having an appropriate size for the desired finger 19 is selected and placed onto the finger 19 securely. Next, the lancet housing or lancet 14 is connected to the port 26 of the holder 12. As discussed previously, the lancet housing or lancet 14 is pushed into the port 26 of the holder 12, such that the engagement portion 56 of the lancet housing or lancet 14 is locked within the locking portion 32 of the holder 12. In this manner, the lancet housing or lancet 14 is securely connected and locked to the holder 12, such that the puncturing element 54 of the lancet housing 14 can be activated to lance or puncture the finger 19. With the lancet 14 connected to the port 26 of the holder 12, the lancet 14 is in communication with the finger 19.

When it is desired to activate the lancet 14 to lance the skin of the finger 19, the lancet 14 is pushed against a finger 19 to activate a retractable mechanism 58 of the lancet 14 to lance the finger 19. After the finger 19 is lanced to create blood flow from the finger 19, the lancet 14 is removed from the holder 12 and the collection container 16 is pushed into the port 26 of the holder 12. With the container 16 properly secured to the holder 12 for collection of a blood sample, the user repeatedly squeezes and releases the wings 38 of the holder 12 to pump and/or extract blood from the finger 19 until a desired amount of blood is collected in the collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

Once a desired amount of blood is collected within the container 16, a blood collector portion including the collection container 16 can be detached from the collection device 10 in order to send a collected sample to a diagnostic instrument and/or testing device. The blood collector portion can be sealed via the cap or septum once removed from the collection device 10 to protectively seal the blood sample within the collection container 16.

### Sample Collection Container Lid and Latch

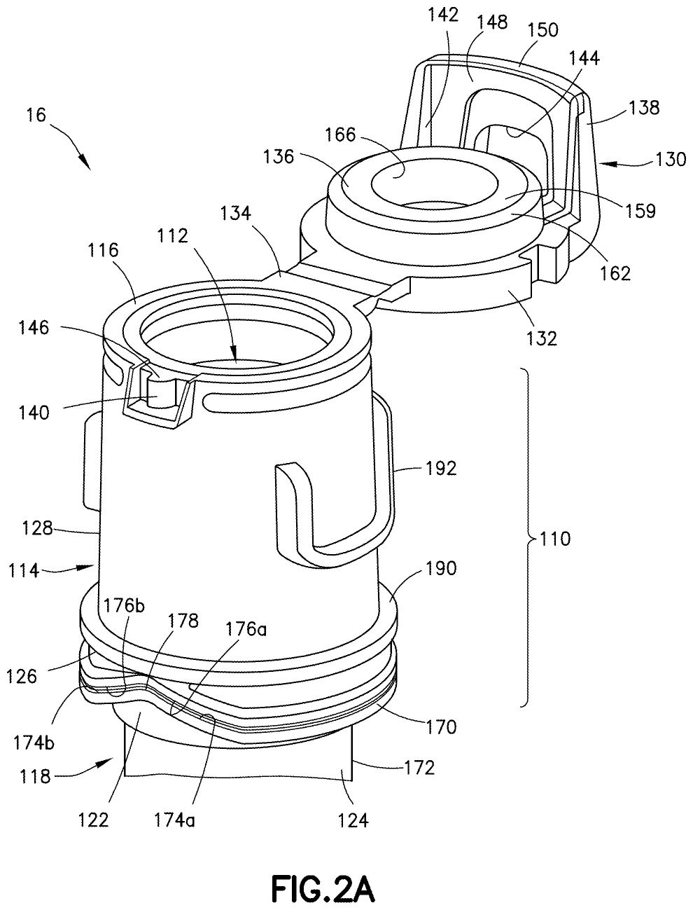

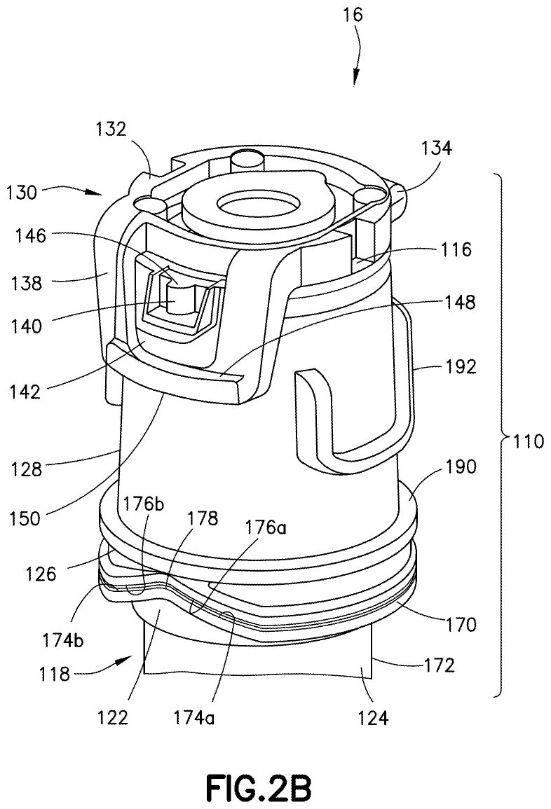

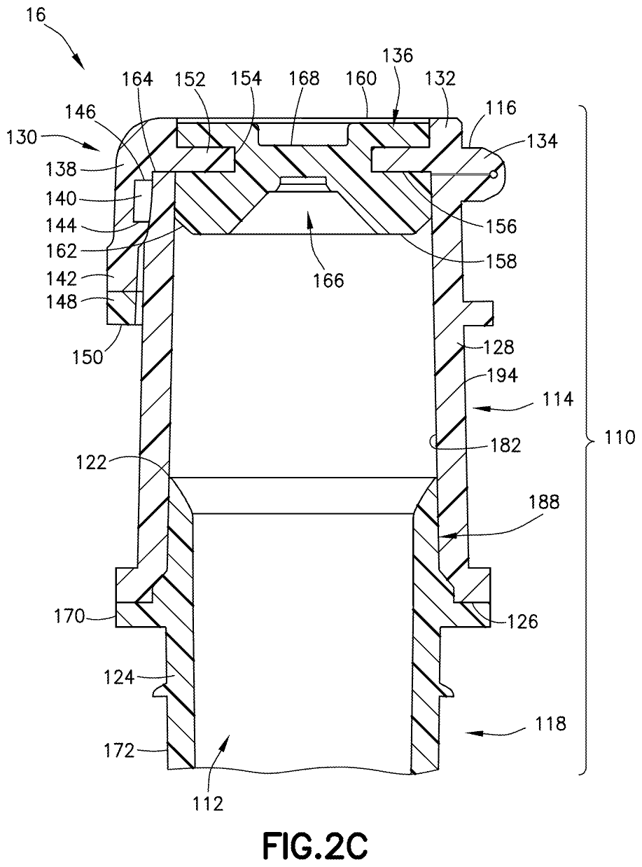

Features of the cap and lid of the collection container 16 are shown in FIGS. 2A-2C. As previously described, the interface between the cap and lid is configured to allow for easy closure (i.e., one handed operation), while still providing a sufficiently robust seal between the cap and the lid to prevent the sample from leaking from the container 16 during, for example, reverse centrifugation.

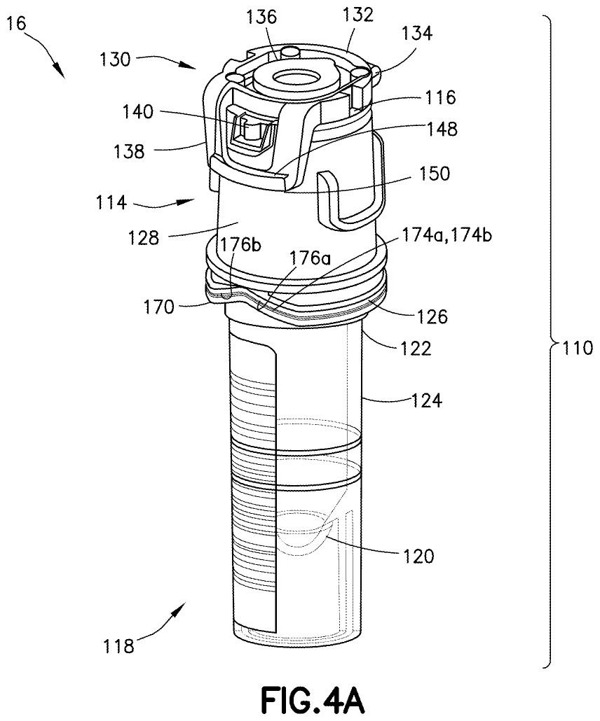

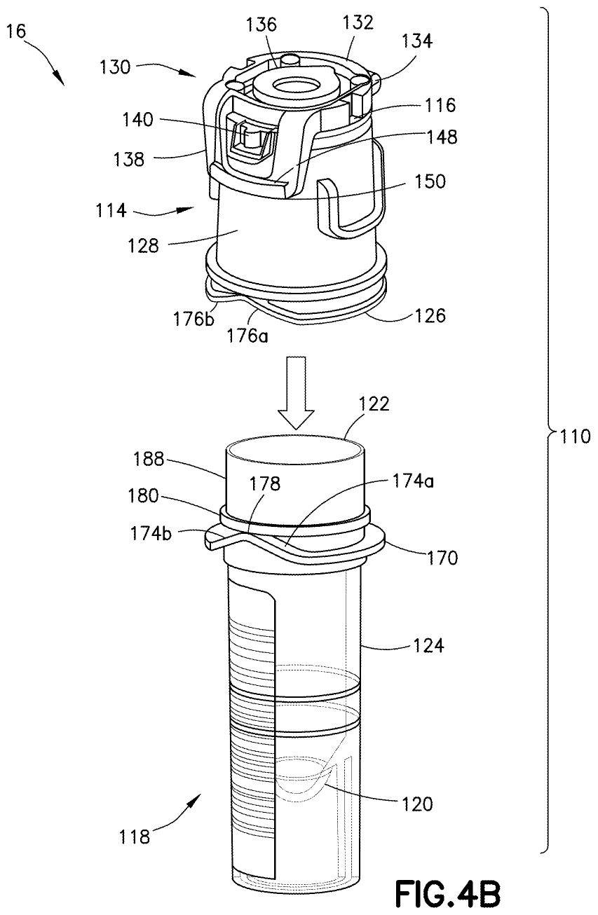

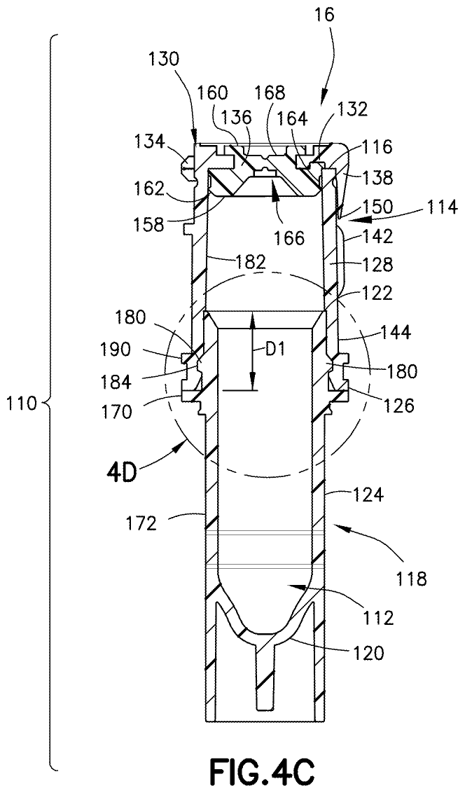

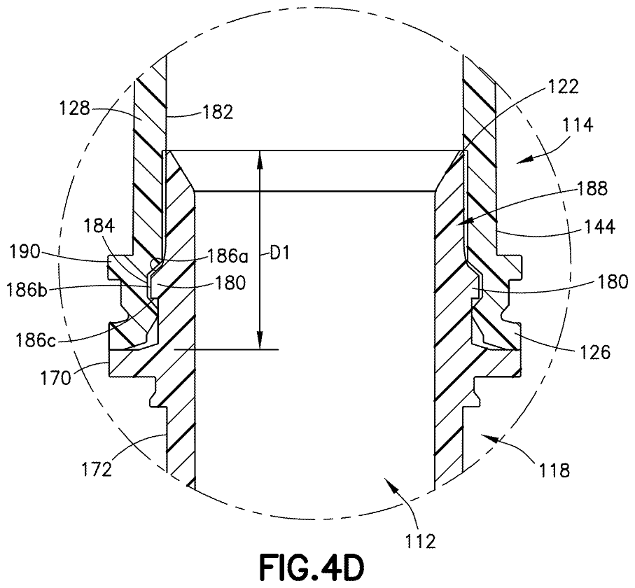

As shown in FIGS. 2A-2C, the blood collection container 16 of the present disclosure includes a container body 110 defining an interior 112 configured to receive the blood sample from the port 26 of the holder 12. The container body 110 includes an upper portion 114 or cap having an open or partially open top 116 and a lower portion 118 (also referred to herein as a base or body) having a closed bottom 120 (shown in FIGS. 4A-4C). In some examples, the upper portion 114 and the lower portion 118 are integrally formed and/or non-removably attached together by, for example, an adhesive or ultrasonic welding.

In other examples, as shown most clearly in FIGS. 4A-4D, the upper portion 114 or cap and the lower portion 118 are separate components that can be removably connected together and can be disassembled by, for example, twisting the upper portion 114 or cap relative to the lower portion 118 and/or by pulling the upper portion 114 or cap axially away from the lower portion 118. In some examples, the lower portion 118 of the container body 110 can include the closed bottom 120, a lower portion open top 122, and a lower portion annular sidewall 124 extending therebetween. Similarly, the upper portion 114 or cap can include an open bottom 126 inserted over the open top 122 of the lower portion 118, the upper portion open top 116, and an upper portion annular sidewall 128 extending therebetween.

The sample collection container 16 further includes a lid 130 including a lid body 132, a flexible connection, such as a hinge 134 or living hinge, a pierceable septum 136, and a latch 138. The flexible connection or hinge 134 is connected between the lid body 132 and the upper portion 114 or cap of the container body 110. The hinge 134 is a flexible member that is configured to open and close, thereby moving the lid 130 between a closed position (shown, for example, in FIG. 2B), in which the lid 130 covers the open top 116 of the upper portion 114 or cap and an open position (shown, for example, in FIG. 2A), where the open top 116 of the upper portion 114 or cap is uncovered. Various structures of flexible connections or hinges 134 that can be formed from rigid or semi-rigid materials will be known to those skilled in the art, which can be adapted for use with the collection container 16 of the present disclosure. For example, the hinge 134 can be an elongate member including one or more notches or scored lines that provide a bend point for the hinge 134. The hinge 134 is configured to pivot or unfold about the bend point, thereby allowing the lid 130 to transition between the closed position and the open position.

As shown most clearly in FIG. 2C, the pierceable septum 136 is mounted to the lid body 132 and seals the open top 116 of the upper portion 114 when the lid 130 is in the closed position. The latch 138 extends from the lid body 132 and, when the lid 130 is in the closed position, engages a protrusion or catch 140 on the upper portion 114 or cap of the container body 110 to secure the lid 130 remains in the closed position.

One benefit of the latch 138 is that it helps to align the open top 116 of the upper portion 114 or cap with the septum 136 even when the user attempts to close the lid 130 with one hand. Thus, the latch 138 beneficially avoids errors caused by improper alignment of the lid 130 and container body 110. The latch 138, septum 136, and flexible connection or hinge 134 of the lid 130 of the present disclosure also contribute to creating the secure engagement between the lid 130 and the container body 110. The present inventors have recognized that the easy closure and secure engagement are needed because the lid 130 of the container 16 can be subjected to several different types of loads during normal use and/or during anticipated or likely misuse of the container 16. The lid 130 and the latch 138 of the present disclosure are configured to overcome such loads so that the lid 130 remains in the closed position protecting the blood sample contained in the container 16. For example, forces may be exerted on the lid 130 and/or the latch 138 during automated handling, where the collection container 16 is dropped (in the upside-down or lid 130 down orientation) a distance of from about 4 inches to about 18 inches into the well of the centrifuge. The collection container 16 may also be subjected to forces during loading, torqueing, and/or impacts during automated handling by blood analyzer instruments. The collection container 16 may also be subjected to accidental drops by users, where a user may drop the collection container 16 a vertical distance of far greater than 18 inches. Forces may also be exerted on the lid 130 and/or latch 138 during loading, unloading, and/or during transport from a sample collection site to a lab. Forces may also be exerted on the lid 130 and/or latch 138 when, for example, an aspiration needle withdraws through the pierceable septum 136, which could pull the lid 130 away from the upper portion 114 or cap of the container body 110. The latch 138 also acts as an anti-tamper system to prevent accidental opening and blood spillage from the collection container 16.

With reference again to FIGS. 2A-2C, in some examples, the latch 138 includes a frame 142 defining an opening 144 that receives the catch 140 of the upper portion 114 or cap, when the lid 130 is in the closed position. For example, the opening 144 can be a square or rectangular shaped opening 144 sized to receive a square or rectangular shaped protrusion of a similar size extending radially outwardly from an outer surface of the sidewall 128 of the upper portion 114 or cap. In some examples, the catch 140 includes a sloped or ramped surface 146, positioned such that as the lid 130 transitions between the open position and the closed position, the latch 138 deflects over the catch 140, assisted by the sloped surface 146, and then snaps back into place to engage the catch 140. The frame 142 of the latch 138, along with the lid body 132 and hinge 134, can be integrally formed by, for example, injection molding from a rigid or semi-rigid material, such as hard plastic (i.e., polypropylene).

The latch 138 also includes a cushion 148 or spacer positioned on or proximate to a leading edge 150 of the frame 142 of the lid 130. The cushion 148 is provided to restrict the latch 138 from being easily and/or inadvertently disengaged from the catch 140. As used herein, a restricting structure, such as the cushion, “restricts” another structure (i.e., the latch 138) from disengaging when additional force, time, or performance of a more complex action is needed to cause the disengagement to occur than if the restricting structure were not present. In particular, for the present disclosure, it is understood that the cushion 148 does not entirely prevent the latch 138 from being disengaged from the catch 140. Instead, as described in further detail herein, the cushion 148 makes it more difficult for a user to disengage the latch 138 from the catch 140 than if the cushion 148 were not present, meaning that accidental release of the latch 138 is less likely to occur.

The cushion 148 can be formed from a flexible material (i.e., a material that is more flexible than the material used to form the frame 142, flexible connection or hinge 134, and/or container body 110). For example, the cushion 148 can be formed from a thermoplastic elastomer. The septum 136 may also be formed from the thermoplastic elastomer, such as liquid silicone rubber (LSR), urethane rubber, latex rubber, and the like. In other examples, the cushion 148 and/or septum 136 can be formed from other flexible, elastomeric materials, such as silicone.

When moving the lid 130 to the closed position, the rigidity of the frame 142 works to secure the latch 138 against the catch 140. In particular, the latch 138 can be configured to work as a cantilever beam, meaning that the latch 138 flexes over the catch 140, and then snaps into place, thereby creating the secure engagement between the latch 138 and the catch 140. The cushion 148 acts as an anti-tamper mechanism. In particular, the cushion 148 on the leading edge 150 of the latch 138 makes it more difficult for the user (i.e., the user's fingers or some unintended removal tool) to gain sufficient leverage to peel-back the latch 138 by blocking any spaces that the user's fingernails or a tool could be inserted to pry the latch 138 open. Beneficially, when the cushion 148 is present, it would be nearly impossible for a user to insert a gloved hand under the latch 138 to gain sufficient leverage to release the latch 138, making inadvertent opening very unlikely to occur in a medical setting. In order to release the latch 138, a purpose-built removal tool can be provided that is sized to be inserted under the cushion 148 to release the latch 138. Providing a lid 130 and latch 138 that can only be easily opened using a specific and specially designed removal tool ensures that the lid 130 is only opened in a controlled and purposeful manner.

Another advantage of the cushion 148 is that it increases flexibility of the latch 138 compared to if no cushion 148 were present. Specifically, because the cushion 148 is present on the latch 138, the frame 142 of the latch 138 can be rather thin without risk of breaking. The combination of the thin frame 142 and flexible cushion 148 means that the latch 138 can be easily bent in a backwards direction (i.e., radially away from the container body 110). Due to this inherent flexibility of the latch 138, during closing, the latch 138 easily deflects over the catch 140, aided by the ramped or sloped surface 146 of the catch 140, and then snaps back into position to engage the catch 140. Accordingly, the cushion 148 also contributes to the easy one-handed closure provided by the lid 130 and latch 138 of the present disclosure.

In some examples, the lid body 132 can include an annular flange 152 defining an opening 154. As shown in FIG. 2C, the septum 136 is inserted through the opening 154 with the annular flange 152 received within an annular notch 156 of the septum 136. The septum 136 is configured to be press fit into the open top 116 of the upper portion 114 or cap of the container body 110, thereby sealing the blood collection container 16 when the lid 130 is in the closed position. As previously discussed, the engagement between the septum 136 and the open top 116 of the upper portion 114 is intended to provide a robust seal sufficient to overcome forces exerted on the septum 136 and/or sample collection container 16 during reverse centrifugation. In order to create the robust seal, an inner diameter of the open top 116 of the upper portion 114 and an outer diameter of the septum 136 are selected to control the initial closure-force that is needed to close the lid 130 and the initial sealing pressure provided by the septum 136. The geometry and inherent flexibility of the septum 136 also contribute to high-pressure sealing properties of the septum 136 during reverse centrifuging. In particular, an initial interference between the open top 116 of the upper portion 114 and the septum 136 (i.e., outer diameter minus inner diameter) is optimized to allow for both an easy closure force and sufficient initial sealing pressure across the tolerance range.

In some examples, the septum 136 can include an inwardly facing surface 158 that faces the interior 112 of the container body 110 and an outwardly facing surface 160 opposite the inwardly facing surface 158. In some examples, the inwardly facing surface 158 of the septum 136 comprises a chamfered peripheral edge 162. As used herein, a “chamfered” edge refers to an edge that is inwardly sloped, angled, or tapered to facilitate insertion of the septum 136 into the container body 110. In some examples, the chamfered edge 162 includes an annular angled surface. In other examples, as shown in FIG. 2C, the chamfered edge 162 is an annular curved surface. Similarly, the upper portion 114 or cap of the container body 110 can include an annular chamfered edge 164 positioned to contact the chamfered peripheral edge 162 of the septum 136 as the lid 130 moves to the closed position. The annular chamfered edge 164 of the upper portion 114 or cap is sized and configured to deform the septum 136 when the lid 130 is in the closed position, thereby sealing the interior 112 of the container body 110.

The chamfered edges 162, 164 can also contribute to the low closure force of the lid 130 and container body 110 interface that allows for one handed closure without requiring any tools. In particular, the chamfered edges 162, 164 are shaped and positioned to easily slide past one another so that minimal pressing force is needed to close the lid 130. The latch 138 also contributes to ensuring that a proper seal is formed by providing visual confirmation that the septum 136 is inserted into the container body 110 to a proper insertion depth. In particular, the user can see and often hear when the opening 144 of the latch 138 snaps onto and/or engages the catch 140.

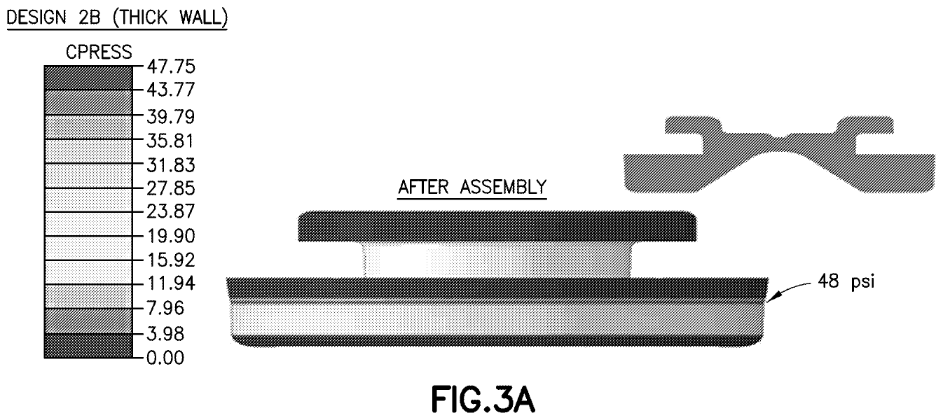

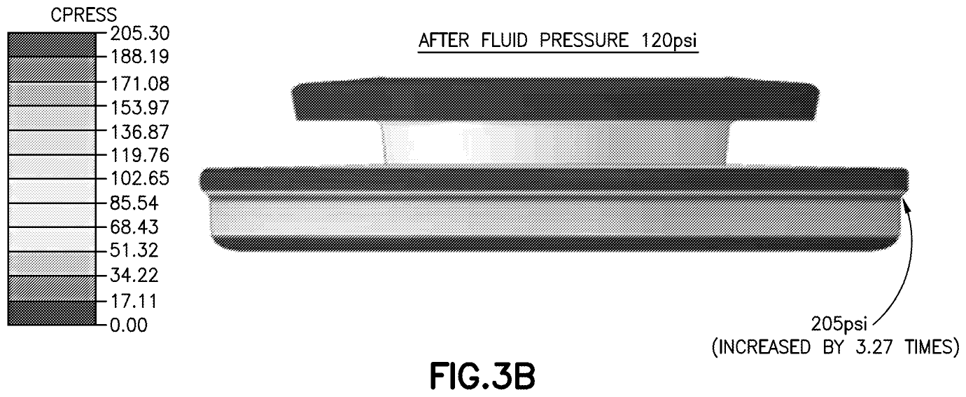

When the septum 136 is inserted into the container body 110 to the proper seal depth, the septum 136 is capable of “mushrooming” when high-pressure forces of reverse centrifugation are exerted on the septum 136. Mushrooming refers to a deformation of the septum 136 where portions of the septum 136 move radially outwardly contacting and pressing against the sidewall 128 of the upper portion 114 to provide an enhanced seal between the septum 136 and container body 110. In some examples, the geometry of the septum 136 also contributes to the mushroom effect that occurs during centrifuging. For example, the inwardly facing surface 158 of the septum 136 can include or define a central cavity 166, which enhances radial deformation of the septum 136 during centrifugation. The central cavity 166 can be any shape that increases or improves radial flexibility of peripheral portions of the septum 136. For example, as shown in FIG. 2C, the cavity 166 includes tapered surfaces, such as a tapered annular surface forming a conical or v-shaped cavity. The conical or v-shaped geometry of the cavity 166 of the septum 136 allows the flexible material of the septum 136 to flex outwardly to increase seal pressure when the septum 136 is exposed to high g-force of reverse centrifugation. Specifically, since the container 16 is positioned in the centrifuge in an upside down or lid first orientation, the fluid contents in the interior 112 of the container body 110 press against the septum 136 due to gravity and the g-forces created during centrifuging. The forces cause peripheral portions of the septum 136 to flex or bend radially outward against the rigid sidewall 128 of the upper portion 114 or cap of the container body 110 causing the septum 136 to adopt a mushroom shape and enhancing the engagement between the septum 136 and the sidewall 128. This mushrooming or bulging of the septum 136 increases the seal pressure, which prevents the sample from leaking from the container 16. The radial expansion of the septum 136 is shown by the finite element analysis models in FIGS. 4A and 4B. As shown in FIGS. 3A and 3B, the scaling force of the septum 136 increases substantially as fluid pressure exerted against the inwardly facing surface 158 of the septum 136 increases. For example, as shown in FIG. 3A, at low pressure (i.e., after assembly) the sealing force of the septum is about 48 psi. As shown in FIG. 3B, when a fluid pressure of 120 psi is applied to the septum 136, the sealing force increases to 205 psi.

With continued reference to FIGS. 2A-2C, the outwardly facing surface 160 of the septum 136 can include a depression or well 168 positioned to collect any fluid that escapes from the interior 112 of the container body 110 during, for example, retraction of a needle through the septum 136. More specifically, the depression or well 168 can be a blood-splatter well. The well 168 can be configured such that any residual blood that escapes during retraction of the aspiration needle is contained in the recessed blood-splatter well 168. Accordingly, the residual blood is not presented as a surface for direct contact, thereby limiting blood exposure within the lab environment.

As known in the art, sealing structures formed from flexible materials, such as thermoplastic elastomers, can become compression-set when held in a stressed condition for extended periods of time. While this may be a disadvantage for a long-term seal, the septum 136 of the present disclosure generally is not expected to be in a stressed condition over most of its usable life. Instead, septum 136 is only expected to be placed under stress during a single use, specifically after the blood specimen is collected and the lid is closed. Thus, it is expected that a total duration that the septum 136 will be in a stressed condition (including during the reverse centrifugation process) is approximately 48 hours. It is believed that the septum 136 will not become compression-set and/or lose sealing ability in this short period of time. Further, it is believed that the material and geometry of the septum 136 of the present disclosure, when used as a single-use product, allows the septum 136 to take advantage of inherent flexibility of a material, such as a thermoplastic elastomer, to provide enhanced sealing for the container 16, especially during reverse centrifugation, without suffering from problems associated with compression set over time.

The flexible material (i.e., the thermoplastic elastomer) of the septum 136 can be readily processed by injection molding. Advantageously, features of the septum 136, such as the well 168, the cavity 166, and the notch 156, are formed without undercuts, allowing for simple injection molding for production. Further, the injection molded material can create a bond with a semi-rigid substrate, such as with the lid body 132, thereby joining the lid body 132 and the septum 136 together. Such joining of the septum 136 and lid body 132 gives a robust and easy-to-produce component that can be produced in a high-volume capacity, with little-to-no assembly. While other materials may also be used for formation of the lid body 132 and/or septum 136, lids 130 formed from most other materials may require assembly and/or bonding of the rigid/flexible parts, resulting in more costly production processes. For example, a silicone material would give excellent flexibility and exhibit less compression-set behavior than the thermoplastic elastomer materials described herein. However, a silicone septum 136 would be more difficult to produce by injection molding because silicone does not create a bond with a lid body 132 or frame 142 formed from polypropylene.

Another benefit of the sample collection container 16 of the present disclosure is that the septum 136 is integrated with the lid 130. Similarly, the lid 130 and the latch 138 are integrated with the container body 112 through the hinge 134. This construction allows for a single component configuration, which is believed to be a benefit compared to devices or assemblies formed from multiple loose components. In particular, loose components can be lost or dropped by the user, or (in the case of an O-Ring) would require additional assembly steps during manufacture. Also, an O-ring would not allow for increased seal-pressure during reverse centrifugation, as provided by the septum 136 of the present disclosure.

While sealing between the lid 130 and the container body 110 could also be achieved using two rigid materials (i.e., surface to surface contact between opposing polypropylene structures of the lid 130 and the container body 110), such engagements between rigid materials would either require high closure-force or a closure-method, such as threads or a bayonet-connection, to give a mechanical advantage to the user to force the lid 130 to the closed position. If screw threads were used, the lid 130 would need to be free-spinning relative to the container body. In that case, the lid 130 would need to be a separate component from the container body 110. While the lid 130 could be attached to the container body 110 by, for example, a tether, such an arrangement would be more difficult to assemble than the integrated container 16 of the present disclosure. Accordingly, the present inventors believe that the container 16 of the present disclosure, which includes the integrated lid 130, septum 136, and latch 138, provides advantages over other types of lids and closures, as may be known in the art.

Sample Collection Container with Separable Container Body

With reference to FIGS. 4A-4D, the upper portion 114 or cap of the container body 110 can be separable from and/or removably attached to the lower portion 118 of the container body 110. A connection interface between the upper portion 114 or cap and the lower portion 118 can be configured to provide an easy guided assembly (i.e., an axial press-fit connection), radial orientation of the components (i.e., radial orientation of the upper portion 114 or cap relative to the lower portion 118), a secure liquid-tight and air-tight sealing between the portions 114, 118, and controlled dis-assembly during de-capping using a specialized cap removal tool.

As shown in FIGS. 4C and 4D, the upper portion 114 or cap is sized to be press fit over the lower portion 118 with an overlap distance D1. As used herein, the “overlap distance” refers to the maximum distance that the upper portion 114 or cap overlaps the top 122 of the lower portion 118, when the upper portion 114 or cap is fully seated on the lower portion 118. In this fully seated position (i.e., at the overlap distance D1), the upper portion 114 is restricted from rotation relative to the lower portion 118. As used herein, a first structure is “restricted” from rotation relative to a second structure when some component of the first structure and/or the second structure makes rotation of the structures relative to one another more difficult than if the component were not present. For example, the first structure and/or the second structure may include protrusions, recesses, detents, angled surfaces, screw treads, and any other components that make rotation of one structure relative to the other structure more difficult. While it may still be possible for a user to rotate the first structure relative to the second structure, sufficient force is required to overcome any engagement between the first structure and the second structure provided, for example, by the protrusions, recesses, detents, angled surfaces, screw treads, or other components.

The upper portion 114 or cap and the lower portion 118 of the container body 110 are intended be securely connected together to protect the blood sample in the interior 112 of the container body 110. Further, the present inventors believe that the connection or interface between the portions 114, 118 needs to be sufficiently secure and robust enough to withstand foreseeable misuse without allowing leakage or separation of the upper portion 114 or cap from the lower portion 118 at inappropriate or unexpected times. At appropriate times, the connection or interface between the upper portion 114 or cap and the lower portion 118 should be easy to overcome in a controlled manner, thereby allowing users to easily access the blood sample contained in the interior 112 of the container body 110. As described in further detail hereinafter, this robust and secure connection is achieved by a tapered interference-fit seal connection in combination with a cam or ramp surface to give mechanical advantage for separation of the upper portion 114 or cap from the lower portion 118. Beneficially, the connection or joint interface of the present disclosure is expected to be easy to assemble and includes components for control of radial orientation. The upper portion 114 or cap and lower portion 118 also do not separate easily, meaning that it would be difficult for a user to inadvertently separate the upper portion 114 or cap from the lower portion 118 at inappropriate times.

In some examples, as shown in FIGS. 4A-4D, the lower portion 118 of the container body 110 includes an annular flange 170 extending radially outwardly from an outer surface 172 of the annular sidewall 124 of the lower portion 118. The annular flange 170 can be configured such that when the upper portion 114 is inserted onto the lower portion 118, the open bottom 126 of the upper portion 114 or cap contacts the annular flange 170 of the lower portion 118 to restrict rotation of the upper portion 114 relative to the lower portion 118. In some examples, rotation of the upper portion 114 or cap relative to the lower portion 118 is restricted by an engagement between cam or ramp surfaces 174a, 174b (shown in FIGS. 4A-4C) on the annular flange 170 and corresponding cam or ramp surfaces 176a, 176b (shown in FIGS. 4A-4C) on the bottom 126 of the upper portion 114 or cap. The ramp surfaces 174a, 174b, 176a, 176b are also configured to assist in controlling radial orientation of the upper portion 114 or cap relative to the lower portion 118 during assembly.

More specifically, in some examples, the annular flange 170 extending from the lower portion 118 includes a first ramp surface 174a angled in a first direction and a second ramp surface 174b angled in a second direction. The bottom 126 of the upper portion 114 or cap includes corresponding ramp surfaces, such as a first ramp surface 176a and a second ramp surface 176b, configured to be axially aligned with the ramp surfaces 174a, 174b of the lower portion 118 when the upper portion 114 is inserted onto the lower portion 118. In some examples, the first ramp surface 174a and the second ramp surface 174b are adjacent to each other, creating a crown or peak 178 pointing towards the top 122 of the lower portion 118. The ramp surfaces 176a, 176b on the bottom 126 of the upper portion 114 also form a peak positioned to align with the peak 178 of the annular flange 170. In some examples, the annular flange 170 and bottom 126 of the upper portion 114 or cap can include multiple peaks 178, such as two peaks positioned on opposite sides of the annular flange 170. In other examples, the annular flange 170 and bottom 126 of the upper portion 114 or cap can include more than two peaks, such as four peaks located at the 0 degree, 90 degree, 180 degree, and 270 degree positions around the annular flange 170 and/or bottom 126.

In some examples, the ramp surfaces 174a, 174b, 176a, 176b are positioned not only to restrict rotation of the portions 114, 118, but also to assist in separating the upper portion 114 or cap from the lower portion 118 of the container body 110. For example, the ramp surfaces 174a, 174b, 176a, 176b can be positioned such that rotation of the upper portion 114 or cap relative to the lower portion 118 by a small amount (i.e., a quarter turn) causes the ramp surface 176a, 176b of the upper portion 114 to slide along the ramp surface 174a, 174b of the annular flange 170 of the lower portion 114, thereby lifting the upper portion 114 from the lower portion 118. Thus, the ramp surfaces 174a, 174b, 176a, 176b provide a mechanical advantage to control dis-assembly during de-capping. Specifically, with the lower portion 118 of the container body 110 fixed, rotation of the upper portion 114 or cap causes the ramp surfaces 174a, 174b to push the upper portion 114 or cap axially away from the lower portion 118 beyond any axial retention features, thereby releasing the upper portion 114 from the lower portion 118. Once the upper portion 114 is moved beyond any axial retention features, the upper portion 114 or cap can be fully removed with very low force, such as by grasping the upper portion 114 or cap and moving it axially away from the container body 110.

Accordingly, it is understood that the upper portion 114 or cap is not completely free of the lower portion 118 after rotating the upper portion 114 or cap by the small amount (i.e., the quarter-turn rotation). Instead, the rotation by the small amount releases the upper portion 114 or cap from the lower portion 118 so that it can be easily separated even, for example, by a user wearing gloves (i.e., with a gloved fingertip force). Further, due to the relatively simple release technique, it is believed that fluid contents in the container 16 (i.e., the blood sample) will not be disturbed during this release action from, for example, jerking or abrupt motions, which could cause spillage.

It is recognized that the upper portion 114 or cap could also be connected to the lower portion 118 by other connection interfaces as are known in the art, such as by a threaded connection or a luer lock connection. However, while a common threaded connection would join and seal the interface between the upper portion 114 or cap and the lower portion 118, a common threaded connection would not allow for controlled orientation of the upper portion 114 or cap relative to the lower portion 118. Also, a common threaded connection is not as easy for installation in a high-speed-assembly production environment compared to the axial press-fit connection of the present disclosure. Further, a common threaded connection could be easily defeated by the user at an improper time during the product's use-life. In contrast, the connection interface between the upper portion 114 or cap and the lower portion 118 of the present disclosure allows for simple assembly in high-speed automated production and a robust connection that is not easily defeated by the user.

In some examples, engagement between the upper portion 114 or cap and the lower portion 118 of the container body 110 can be further enhanced by protrusions, extended portions, textured regions, and/or similar protruding structures extending from surfaces of the sidewall of the upper portion 114 or cap and/or from the sidewall of the lower portion 118. For example, as shown in FIGS. 4C and 4D, the lower portion 118 of the container body 110 can include an annular protrusion 180 extending radially outward from the outer surface 172 of the annular sidewall 124 of the lower portion 118. An inner surface 182 of the upper portion sidewall 128 can include an annular recess 184 that receives the annular protrusion 180 of the lower portion 118. The annular protrusion 180 can be shaped to make it easier to slide the upper portion 114 or cap onto the lower portion 118. For example, the annular protrusion 180 of the lower portion 118 can include a sloped upper surface 186a, a substantially planar lower surface 186b, and a cylindrical peripheral edge 186c extending therebetween. The inner surface 182 of the upper portion 114 or cap is configured to slide along the sloped upper surface 186a of the annular protrusion 180, pushing the sidewall 128 of the upper portion 114 or cap slightly radially outwardly to facilitate insertion of the lower portion 118 into the upper portion 114.

In some examples, the engagement between the upper portion 114 or cap and the lower portion 118 is further enhanced by providing a slight taper to the lower portion sidewall 124 near the top 122 of the lower portion 118. For example, the sidewall 124 of the lower portion 118 can include a tapered portion 188 that tapers radially outwardly towards the open top 122 of the lower portion 118. The tapered portion 188 can be configured to provide a tapered interference fit between the outer surface 172 of the lower portion sidewall 124 and the inner surface 182 of the upper portion sidewall 128. In order to provide the tapered interference fit, a tapered inner diameter of the upper portion 114 or cap and a tapered upper portion diameter of the lower portion 118 are selected to allow for simple axial press-fit assembly. Further, the geometry of the taper can allow for axial alignment during assembly, guiding the two portions 114, 118 of the container body 110 together.

In some examples, the upper portion 114 or cap may also include one or more stiffener ribs 190 extending from an outer surface 194 of the sidewall 128 of the upper portion 114. For example, the stiffener ribs 190 can be annular ribs or areas of increased thickness that increase overall stiffness of the upper portion 114. Increasing stiffness of the upper portion 114 is believed to provide a more secure connection between the portions 114, 118. The axial-length of the tapered portion 188 or sealing area and the annular flange 170 on the lower portion 118 also help to make the joining interface between the portions 114, 118 more rigid and to prevent wobble. It is believed that at least these features help to stiffen the interface between the portions 114, 118 and to maintain seal-pressure across full shelf-life of the collection container 16, as well as to resist creep and material relaxation over time. In contrast, a simple annular bead/band on one of the portions 114, 118 would provide a seal, but would not give a robust mechanical connection to withstand use/abuse, eliminate wobble, and keep a liquid-tight/air-tight seal. Further, the annular bead/band would be an undercut feature in the injection mold tooling, and would require tight tolerances and/or complex tooling during production compared to providing a simple tapered interference-fit features.

### Removal Tool for Separating Portions of the Container Body

In some examples, the upper portion 114 or cap and/or the lower portion 118 of the container body 110 include features to aid in de-capping using specifically designed or selected tools. For example, the upper portion 114 or cap can include u-shaped ribs 192 (shown in FIGS. 4A-4C) that interface with a tool to give mechanical advantage during the small (i.e., quarter-turn) rotation to release the upper portion 114. The u-shaped ribs 192 can be relatively shallow so that the user is not tempted to grasp the ribs 192 to attempt to release the upper portion 114 using his or her fingers instead of the tool. However, the u-shaped ribs 192 should be of sufficient depth so that when contacted by a specific tool, the u-shaped ribs 192 transmit sufficient torque from the tool to the upper portion 114 or cap to release the upper portion 114 or cap from the lower portion 118. Further, desirably, the tool can be configured to leave the interface between the lid 130 and the top 116 of the upper portion 114 or cap unstressed so as not to break the seal between the septum 136 of the lid 130 and the top 116 of the upper portion 114 or cap during the de-capping operation.

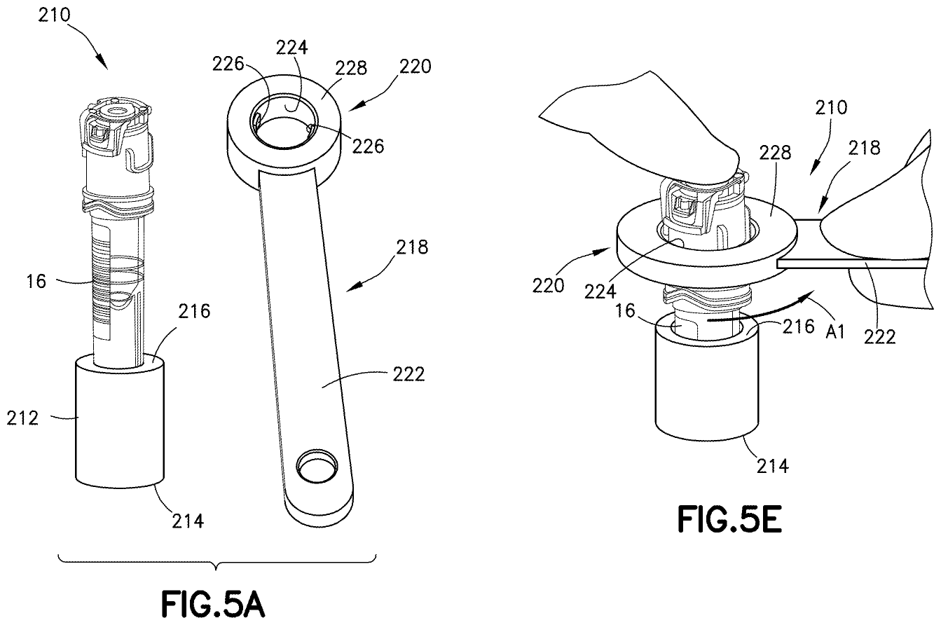

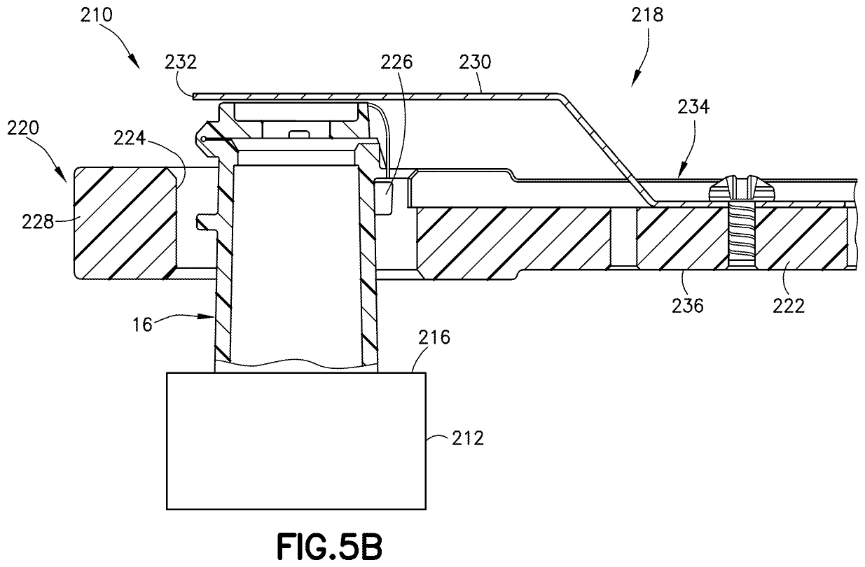

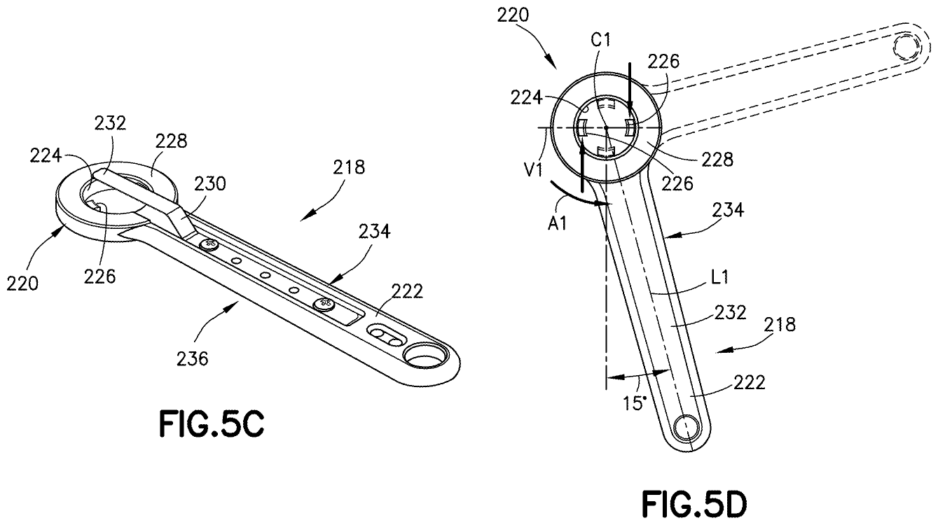

A cap removal assembly 210 including features of the present disclosure for disassembly of the upper portion 114 or cap of a blood collection container 16 from a container body 110 or lower portion 118 is shown in FIGS. 5A-5D. The assembly 210 includes features intended to make the de-capping operation very simple and low-effort, and also to prevent accidental de-capping during use (blood collection and handling).

The assembly 210 includes a stand 212 configured to support the blood collection container 16 in an upright position and to restrict rotation of the container 16 relative to the stand 212. For example, the stand 212 can be a tubular structure including a closed bottom or base 214 and an open top 216. The open top 216 can be sized so that the collection container 16 can be securely inserted into the stand 212 through the open top 216. In some examples, there can also be ribs (not shown) on the bottom 120 of the lower portion 118 of the collection container 16, that (when inserted into the stand 212) engage anti-rotation structures of the stand 212 to resist rotation of the container 16 relative to the stand 212 during de-capping. Desirably, ribs are positioned so that they are not easily accessible to the user, so that the user is unlikely to attempt to grasp them or use them to apply torque to disconnect the upper portion 114 or cap from the lower portion 118. Instead, the ribs on the bottom 120 are desirably positioned so that they are only easily engaged by corresponding structures on the stand 212.

The cap removal assembly 210 also includes a tool 218 having a cap remover 220 and a handle 222 extending therefrom. The cap remover 220 includes a body defining an opening 224 or recess sized to receive a cap or upper portion 114 of the blood collection container 16 and at least one protrusions, such as a prong 226, extending from the body into the opening 224 or recess positioned to engage a portion of the upper portion 114 or cap. For example, the prong 226 can be positioned to engage and/or be inserted into the u-shaped rib 192 of the upper portion 114. Once the tool 218 is engaged to the upper portion 114 or cap, a user can rotate the upper portion 114 or cap relative to the lower portion 118 in a direction of arrow A1 (shown in FIG. 5E) by applying a rotating force to the handle 222 of the tool 218. As previously described, the interface between upper portion 114 or cap and lower portion 118 is configured such that rotating the upper portion 114 or cap by a small amount (i.e., rotation by a quarter turn) releases the upper portion 114 or cap from the lower portion 118. Once the upper portion 114 or cap is released from the lower portion 118 of the container body 110, the upper portion 114 or cap can be removed by lifting the upper portion 114 or cap axially from the lower portion 118.

In some examples, the body of the tool 218 is a ring 228 that defines the opening 224 (i.e., a through-hole or bore extending through the ring 228). In some examples, the cap remover 220 includes two protrusions, such as two prongs 226, extending from an inner surface of the ring 228 into the opening 224. The prongs 226 are positioned and sized to contact the u-shaped ribs 192 on the upper portion 114 or cap of the container 16 to facilitate releasing the upper portion 114 or cap from the lower portion 118. As shown in FIG. 5D, in some examples, the prongs 226 are positioned on opposite sides of the ring 228, such that a virtual line V1 passing between the prongs 226 passes through a center C1 of the opening 224 defined by the ring 228. Further, the prongs 226 can be provided offset from the handle 222 of the tool 218 so that a user can grasp the tool 218 more comfortably. For example, the virtual line V1 between the prongs 226 can be sloped or angled relative to a longitudinal axis LI of the handle 222 by a small amount, such as by about 5 degrees to about 25 degrees, preferably about 15 degrees.

In order to use the cap removal assembly 210 to disconnect the upper portion 114 or cap from the lower portion 118 of the container body 110, the user first places the collection container 16 containing the fluid sample into the stand 212, as shown in FIG. 5A. As previously discussed, mechanical features of the collection container 16 and/or the stand 212 are provided to restrict rotation of the collection container 16 relative to the stand 212. The user next inserts the cap remover 220 of the tool 218 over the upper portion 114 or cap of the collection container 16, moving the cap remover 220 in a downward direction until the prongs 226 are received in the u-shaped ribs 192, as shown in FIG. 5E. Once the cap remover 220 is appropriately positioned, the user can place his or her thumb on the top 116 of the upper portion 114 or lid 130 to hold the collection container 16 in place and to prevent it from being inadvertently lifted out of the stand 212. With the user's thumb on the top 116 of the upper portion 114 or cap, the user rotates the cap remover 220 by a small amount (i.e., by a quarter turn) in the direction of the arrow A1 (in FIG. 5E) by applying a rotation force to the handle 222 of the tool 218, thereby releasing the upper portion 114 or cap from the lower portion 118. Once the upper portion 114 or cap is released from the lower portion 118, the upper portion 114 or cap can be removed by grasping the upper portion 114 or cap and moving it axially away from the lower portion 118.

In some examples, the ring 228 is sized so that the upper portion 114 or cap can be inserted through the opening 224 when the tool 218 is in either an upwardly facing orientation or a downwardly facing orientation. This allows the tool 218 to be used by both right handed and left handed individuals. For example, the tool 218 can be configured to permit right-handed operation when in the upwardly facing orientation, meaning that a right-handed individual inserts the tool 218 over the upper portion 114 or cap with the tool 218 in the upwardly facing orientation. A left handed individual flips the tool 218 over so that the tool 218 is in the downwardly facing orientation and then inserts the cap remover 220 onto the upper portion 114 or cap. By flipping the tool 218 over, the left handed individual can rotate the tool 218 in an opposite direction, which is easier for a left handed individual to perform.

With specific reference to FIGS. 5B and 5C, in some examples, the tool 218 can include a pressing member 230 mounted to the handle 222 and having a distal end 232 extending over the opening 224 of the remover 220. The pressing member 230 is positioned to press against the top 116 of the upper portion 114 or cap of the container 16, meaning that the user does not need to place his or her thumb on the upper portion 114 or cap while rotating the tool 218. As was the case with the user's thumb, the pressing member 230 prevents the container 16 from lifting out of the stand 212 when the tool 218 and the upper portion 114 or cap are rotated relative to the lower portion 118 of the container body 110. The pressing member 230 can be attached to either an upper surface 234 of the tool 218 or to a lower surface 236 of the tool 218, depending on whether the user is right handed or left landed. For example, as shown in FIG. 5C, the pressing member 230 is attached to the upper surface 234 of the tool 218, so that the tool can be engaged to the container 16 in the upwardly facing orientation. In order to reconfigure the tool 218 for use by a left-handed individual, the pressing member 230 should be removed and reconnected to the lower surface 236 of the tool 218, so that the tool 218 can be inserted onto the container 16 in the downwardly facing (i.e., left-handed) orientation.

While different examples of the sample containers, blood collection devices, and associated tools are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A sample collection container configured to be removably mounted to a blood collection device, the collection container comprising:
a container body defining an interior configured to receive a sample provided from the blood collection device, the container body comprising an upper portion comprising an open top and a lower portion comprising a closed bottom; and
a lid comprising:
a lid body;
a flexible connection between the lid body and the upper portion of the container body that moves the lid between a closed position, in which the lid covers the open top of the upper portion, and an open position, where the open top of the upper portion is uncovered;
a pierceable septum mounted to the lid body that seals the upper portion open top when the lid is in the closed position; and
a latch extending from the lid body that engages a catch on the upper portion of the container body to secure the lid in the closed position.

2. The collection container of claim 1, wherein the latch comprises a frame formed from a first material and a cushion formed from a second material that is less rigid than the first material.

3. The collection container of claim 2, wherein the cushion is positioned proximate to a leading edge of the frame of the lid and is configured to restrict the latch from being disengaged from the catch.

4. The collection container of claim 2, wherein the frame comprises an opening that receives the catch of the upper portion when the lid is in the closed position, and wherein the cushion is positioned on the frame between the opening and the leading edge of the frame.

5. The collection container of claim 2, wherein the lid body, the flexible connection, and the container body comprise the first material, and wherein the septum comprises the second material.

6. The collection container of claim 2, wherein the first material is a semi-rigid plastic, and the second material is a thermoplastic elastomer.

7. The collection container of claim 6, wherein the first material is polypropylene, and the second material is at least one of liquid silicone rubber, urethane rubber, and latex rubber.

8. The collection container of claim 1, wherein the catch on the upper portion of the container body comprises a sloped surface positioned such that, as the lid transitions between the open position and the closed position, the latch deflects over the catch prior to engaging the catch.

9. The collection container of claim 1, wherein the septum comprises an inwardly facing surface that faces the interior of the container body and an outwardly facing surface opposite the inwardly facing surface, the outwardly facing surface comprising a recess or well positioned to collect any fluid that escapes from the interior of the container body during retraction of a needle through the septum.

10. The collection container of claim 1, wherein the lid body comprises an annular flange defining an opening and wherein the septum is inserted through the opening with the annular flange received within an annular notch of the septum.

11. The collection container of claim 1, wherein the septum comprises an inwardly facing surface that faces the interior of the container body when the lid is in the closed position, the inwardly facing surface of the septum comprising a chamfered peripheral edge.

12. The collection container of claim 11, wherein the inwardly facing surface of the septum defines a central cavity, which enhances radial deformation of the septum during centrifugation.

13. The collection container of claim 12, wherein the central cavity comprises a tapered annular surface.

14. The collection container of claim 1, wherein the upper portion open top comprises an annular chamfered edge positioned to contact a chamfered peripheral edge of the septum as the lid moves to the closed position.

15. The collection container of claim 14, wherein the annular chamfered edge of the upper portion deforms the septum when the lid is in the closed position, thereby sealing the interior of the container body.

16. The collection container of claim 15, wherein the septum is configured to radially expand during centrifugation, thereby enhancing the sealing of the interior of the container body.

17. The collection container of claim 1, wherein the lower portion of the container body and the upper portion of the container body are separable and removably attached together.

18. The collection container of claim 17, wherein the upper portion is sized to be press fit over the lower portion and, when fully seated on the lower portion, the upper portion is restricted from rotation relative to the lower portion.

19. The collection container of claim 1, wherein the lower portion of the container body comprises the lower portion closed bottom, a lower portion open top, and a lower portion annular sidewall extending therebetween, and
wherein the upper portion of the container body comprises an upper portion open bottom inserted over the lower portion open top of the container body, the upper portion open top, and an upper portion annular sidewall extending therebetween.

20. The collection container of claim 19, wherein the lower portion of the container body comprises an annular flange extending radially outwardly from an outer surface of the lower portion annular sidewall, and wherein, when the upper portion is inserted onto the lower portion, the upper portion open bottom contacts the annular flange of the lower portion to restrict rotation of the upper portion relative to the lower portion.

21. The collection container of claim 20, wherein the annular flange extending from the lower portion and the upper portion open bottom comprise corresponding ramp surfaces configured to be axially aligned when the upper portion is inserted onto the lower portion.

22. The collection container of claim 21, wherein rotation of the upper portion relative to the lower portion causes the ramp surface of the upper portion to slide along the ramp surface of the annular flange of the lower portion, thereby lifting the upper portion off of the lower portion.

23. The collection container of claim 21, wherein the lower portion comprises an annular protrusion extending radially outward from the outer surface of the lower portion sidewall, and wherein an inner surface of the upper portion sidewall comprises an annular recess that receives the annular protrusion of the lower portion.

24. The collection container of claim 23, wherein the annular protrusion extending from the lower portion comprises a sloped upper surface, a substantially planar lower surface, and a cylindrical peripheral edge extending therebetween.

25. The collection container of claim 21, wherein an inner surface of the lower portion sidewall comprises a tapered portion that tapers radially inwardly from the lower portion open top.

26. The collection container of claim 25, wherein there is tapered interference fit between the outer surface of the lower portion sidewall and the inner surface of the upper portion sidewall.

27. The collection container of claim 1, wherein the flexible connection comprises a hinge.

28-37. (canceled)
